(19) United States
(12) Patent Application Publication
(10) Pub. No.: US 2008/0195087 A1
(43) Pub. Date: Aug. 14, 2008

(54) APPARATUS FOR LASER INTERACTION WITH BIOLOGICAL TISSUES

(75) Inventors: Sean Xiaolu Wang, Wilmington, DE (US); Brian Pryor, Ewing, NJ (US)

Correspondence Address: FRANK F. TIAN, 331-4A THIRD AVENUE, LONG BEACH, NJ, 07740, US

(73) Assignee: BWT Property, Inc., Newark, DE (US)

(21) Appl. No.: 12/029,515

(22) Filed: Feb. 12, 2008

Related U.S. Application Data
(60) Provisional application No. 60/889,388, filed on Feb. 12, 2007.

(51) Int. Cl. A61B 18/24

(52) U.S. Cl. 606/15

(57) ABSTRACT

A catheter apparatus is disclosed for controlling the interaction of a fiber coupled treatment laser with biological tissues, where the fiber tip is anchored into the catheter apparatus to keep the fiber tip from direct contact with the biological tissue. The catheter apparatus prevents fiber tip contamination by the biological tissue. The catheter may incorporate temperature sensitive elements for tissue temperature monitoring and control.

### REFERENCE TO RELATED APPLICATIONS

This application claims an invention which was disclosed in Provisional Patent Application No. 60/889,388, filed Feb. 12, 2007, entitled “Apparatus for Laser Interaction with Biological Tissues.” The benefit under 35 USC §119(e) of the above mentioned United States Provisional Applications is hereby claimed, and the aforementioned application is hereby incorporated herein by reference.

### FIELD OF THE INVENTION

This invention generally relates to a catheter apparatus, and more specifically to a catheter apparatus for laser interaction with biological tissues.

### BACKGROUND

Laser can be used to interact with biological tissue for ablation, vaporization, excision, incision, and coagulation purposes. Traditionally, there are two approaches of applying laser light to biological tissue, i.e. contact and non-contact approaches. In the non-contact approach, a CO₂ laser is usually used due to its good beam collimation property. In the contact approach, a diode laser delivered via an optical fiber is generally used. Due to the divergence of the laser beam out of the fiber, the power density decreases drastically within a short distance away from the fiber tip. Thus the fiber tip has to be in contact with the tissue for performing ablation, vaporization, excision, incision, and coagulation. Occasionally, for photo-coagulation and hemostats which requires at a low power density, the fiber tip is kept a small distance away from the tissue or blood vessels.

One of the problems associated with the contact laser operation is the contamination of fiber tip. During the operation, some of the tissue may get attached to the fiber tip due to the tip's sharp cleaved edge and block the laser beam. The tissue will be burned and carbonized, which will further block and absorb the laser energy causing the tip temperature to increase. The hot fiber tip will cause burn when it touches the tissue and result in char. The excess char is highly un-desirable which will cause excessive pain and delay any healing process. In addition, the fiber tip contamination may also damage the fiber tip itself. For some medical procedures and applications, laser energy is used to cause modest photo-coagulation or de-nature for the purpose of hemostat, killing topical bacteria, tissue surface preparing, sterilization etc. For these applications, it is critical to make sure that no excessive damage is induced to the tissue.

Another challenge associated with the contact operation is to kept a relative constant power density for photo-coagulation and de-nature when the hot fiber tip has to be kept at a distance away from the tissue to prevent direct contact. It is difficult to manually keep the fiber tip at a constant distance without using any kind of anchor.

### SUMMARY OF THE INVENTION

The present invention discloses a catheter which is attached to the optical fiber tip with the fiber tip positioned within the catheter. The catheter can be either close ended or open ended. The catheter may be transparent in the wavelength of the laser light that is used in the treatment. The catheter may be made by polymer or any other appropriate material. For the open ended catheter, the optic fiber is anchored a distance away from the tissue with an open air gap in between. For the closed ended catheter, the optic fiber is anchored a distance away from the tissue with the catheter material in between. The end profile of the catheter can be varied to shape the laser beam for different applications. The smooth surface of catheter can further prevent attachment of tissue to the catheter.

The catheter may further incorporate temperature sensitive elements at suitable locations, in which the optical spectral properties of the elements will change under different temperatures. On one hand, this spectral change can be utilized for temperature monitoring in order to control the laser operation procedure. On the other hand, the spectral change can be utilized for automatically controlling the laser power that is delivered to the tissue.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates a first exemplified structure of an open ended laser catheter in accordance with the present invention;

FIG. 2 illustrates a second exemplified structure of a closed ended laser catheter in accordance with the present invention; and

FIG. 3 illustrates a laser catheter with temperature sensitive elements in accordance with the present invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a catheter apparatus for laser interaction with biological tissues. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

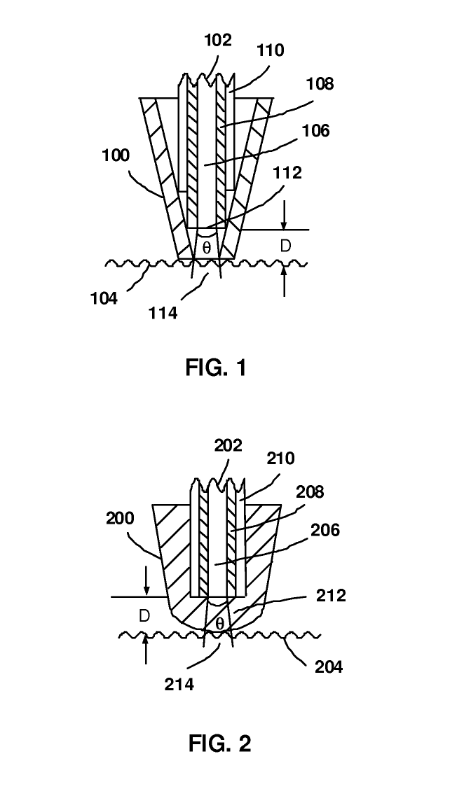

Referring to FIG. 1, the structure of an exemplary open ended laser catheter is disclosed, where an optical fiber tip 102 is anchored into or within an open ended catheter 100 for protection and light intensity control. The fiber tip 102 comprises a core member 106, a cladding member 108, and a jacket member 110, where the laser light is guided in the core member 106. The divergence angle, θ of the laser beam 114 that emits from the end of the fiber tip 112 is determined by the numerical aperture (NA) of the optical fiber. When the catheter 100 is placed in contact with the tissue 104, the end of the fiber tip 112 will be separated from the tissue 104 by an open air gap with a thickness of D because part of catheter 100 is structurally interposed there between. The spot size (A) of the laser beam on the target tissue 104 can be estimated as: A=π·(D·tan(θ/2))̂2. Thus, the light intensity (I) of the laser beam will be maintained at a constant level of: I═P/A, where P is the power of the laser light. In the meantime, since the end of the fiber tip 112 does not touch the tissue 104 directly, tissue debris will have less or virtually no chance to stick onto the fiber tip 102 to block the laser beam and cause contaminate/damage to the fiber tip 102. Cooling air or fluid (not shown) may be supplied through the open ended catheter 100 to assist tissue temperature control.

In another preferred embodiment of the present invention as shown in FIG. 2, a closed ended laser catheter 200 is used for fiber tip protection and light intensity control. In this embodiment, an optical fiber tip 202 is embedded into or within the closed ended catheter 200 and hermetically sealed therein. The fiber tip 202 comprises a core member 206, a cladding member 208, and a jacket member 210, where the laser light is guided in the core member 206. The divergence angle, θ of the laser beam 214 that emits from the catheter 200 is determined by the numerical aperture of the optical fiber as well as by the refractive index, thickness (D), and shape of the distal end 212 of the catheter 200. When the catheter 200 is placed in contact with the tissue 204, the intensity (I) of the laser beam 214 will be maintained at a constant level. In addition, the spatial intensity distribution of the laser beam can be modified by controlling the refractive index, thickness (D), and shape of the distal end 212 of the catheter 200 to adapt for different types of laser operations. The smooth surface at the end of the catheter 212 can further prevent attachment of tissue to the catheter.

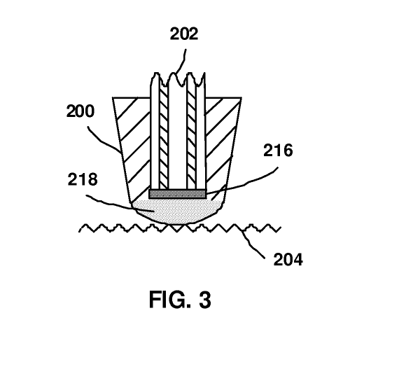

Referring to FIG. 3, the laser catheter 200 can further incorporate temperature sensitive elements whose optical spectral properties will change under different temperatures. Such temperature sensitive element can be dopants 218 within the laser catheter structure 200 or a membrane 216 inserted or formed between the fiber tip 202 and the catheter 200. Any materials that exhibit certain change in their absorption, transmission, reflection, fluorescence or Raman spectrum with temperature can be utilized as the temperature sensitive element. An example of the dopant 218 material is Alexandrite, whose fluorescence lifetime decreases with increased temperature. An example of the membrane material 216 is liquid crystal or thin film filter, whose transmission spectrum will change with temperature. On one hand, this spectral change can be utilized for temperature monitoring in order to control the laser operation procedure. When the measured temperature of the surrounding tissue 204 reaches dangerous or undesirable levels, a warning signal can be sent to the operator to shut down the laser. On the other hand, the spectral change can be utilized for automatically controlling the laser power that is delivered to the tissue 204. For example, a liquid crystal or thin film filter based membrane 216 can exhibit reduced transmittance at the laser wavelength when its temperature rises above certain level. As a result, when the tissue and catheter temperature increases to or above a threshold level, the laser power that is delivered through the membrane 216 will automatically decrease to prevent further tissue temperature increasing.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A catheter comprising:
an outer housing having a distal end; and
a fiber tip disposed within a proximity about the distal end of the outer housing such that a gap with a predetermined thickness is formed between the fiber tip and the distal end.

2. The catheter of claim 1, wherein the outer housing is hermetically sealed with the distal end having the fiber tip embedded within the catheter.

3. The catheter of claim 1, wherein the outer housing comprises temperature sensitive elements.

4. The catheter of claim 1, wherein the distal end of the outer housing has a smooth surface.

5. The catheter of claim 1, wherein the catheter comprises an optical fiber for delivering laser light from a laser source positioned at a proximal end of the optical fiber to a distal end of the optical fiber having the fiber tip.

6. The catheter of claim 5, wherein the distal end of the outer housing contacts biological tissues for controlling laser interaction with biological tissues.

7. The catheter of claim 5, wherein the outer housing is transparent at or about a wavelength of the laser source.

8. The catheter of claim 5, wherein a refractive index, thickness, and shape of the distal end of the outer housing is utilized to control an intensity distribution of the laser light emitting from the distal end of the outer housing.

9. A method for making a catheter comprising the steps of:
providing an outer housing having a distal end; and
providing a fiber tip disposed within a proximity about the distal end of the outer housing such that a gap with a predetermined thickness is formed between the fiber tip and the distal end.

10. The catheter of claim 9, wherein the outer housing is hermetically sealed with the distal end having the fiber tip embedded within the catheter.

11. The method of claim 9, wherein the outer housing comprises temperature sensitive elements.

12. The method of claim 9, wherein the distal end of the outer housing has a smooth surface.

13. The method of claim 9, wherein the catheter comprises an optical fiber for delivering laser light from a laser source positioned at a proximal end of the optical fiber to a distal end of the optical fiber having the fiber tip.

14. The method of claim 13, wherein the distal end of the outer housing contacts biological tissues for controlling laser interaction with biological tissues.

15. The method of claim 13, wherein the outer housing is transparent at or about a wavelength of the laser source.

16. The method of claim 13, wherein a refractive index, thickness, and shape of the distal end of the outer housing is utilized to control an intensity distribution of the laser light that emits from the distal end of the outer housing.
